(19) United States
(12) Patent Application Publication
(10) Pub. No.: US 2026/0232567 A1
(43) Pub. Date: Aug. 13, 2026

(54) A COSMETIC COMPOSITION

(71) Applicant: CONOPCO, INC., D/B/A UNILEVER, Cliffs, NJ (US)

(72) Inventors: Chengdong JI, Shanghai (CN); Jinfang WANG, Shanghai (CN); Shangchun YI, Shanghai (CN); Xinran ZHOU, Shanghai (CN)

(73) Assignee: CONOPCO, INC., D/B/A UNILEVER, Hoboken, NJ (US)

(21) Appl. No.: 19/155,885

(22) PCT Filed: Feb. 5, 2024
(86) PCT No.: PCT/CN2024/076035

§ 371 Date: Aug. 12, 2025

(30) Foreign Application Priority Data
Feb. 17, 2023 (WO) PCT/CN2023/076754
Mar. 14, 2023 (EP) 23161640.0

(51) Int. Cl. A61K 8/85; A61K 8/35; A61K 8/37; A61K 8/40; A61K 8/49; A61Q 17/04

(52) U.S. Cl. CPC A61K 8/85; A61K 8/35; A61K 8/37; A61K 8/40; A61K 8/4946; A61Q 17/04

(57) ABSTRACT

Disclosed is a cosmetic composition comprising polyhydroxyalkanoate and 0.1 to 25% of organic sunscreen by weight of the composition, wherein the weight ratio of the polyhydroxyalkanoate to the organic sunscreen is at least 1:2.

### FIELD OF THE INVENTION

The present invention relates to a cosmetic composition comprising polyhydroxyalkanoate and 0.1 to 25% of organic sunscreen by weight of the composition, wherein the weight ratio of the polyhydroxyalkanoate to the organic sunscreen is at least 1:2. It was surprisingly found that the composition is capable of providing improved sun protection factor (SPF).

### BACKGROUND OF THE INVENTION

Solar radiations include about 5% ultraviolet (UV) radiation, wavelength of which is between 200 nm and 400 nm. It is further divided into three regions: from 320 to 400 nm (UV-A), 280 to 320 nm (UV-B) and from 200 to 280 nm (UV-C). Substantial portion of UV-C radiation is absorbed by ozone. Scientific studies have indicated that exposure to UV-A and UV-B radiation for short period causes pigmentation, reddening of the skin and localized irritation, whereas continued or prolonged exposure can lead to sunburn, formation of wrinkles and age spots. UV radiation is also thought to cause significant damage to hair. Therefore, it is desirable to protect the skin and other keratinous substrates of the human body from the harmful effects of both UV-A and UV-B radiation.

This is often delivered through personal care compositions e.g. cosmetic compositions, which provide protection from ultraviolet (UV) radiation. Organic sunscreens e.g. 4-tert-butyl-4′-methoxydibenzoylmethane (BMDM) absorb the UVA radiation. The ability of a sunscreen to provide protection by absorbing UVA radiation is expressed in terms of UVA protection factor (UVAPF). On the other hand, organic sunscreens, e.g. 2-ethylhexyl 2-cyano-3,3-diphenylacrylate (OCR) and octyl methoxycinnamate (MCX) absorb the UVB radiation. The ability of a sunscreen to provide protection by absorbing UVB radiation is expressed in terms of sun protection factor (SPF).

Despite efforts so far, the present inventors have recognized that there are high needs to provide cosmetic composition with improved SPF. It was surprisingly found that when including a polyhydroxyalkanoate and 0.1 to 25% of organic sunscreen by weight of the composition into a cosmetic composition with a weight ratio of polyhydroxyalkanoate the organic sunscreen is at least 1:2, the composition is capable of providing improved SPF.

### SUMMARY OF THE INVENTION

In a first aspect, the present invention is directed to a cosmetic composition comprising polyhydroxyalkanoate and 0.1 to 25% of organic sunscreen by weight of the composition, wherein the weight ratio of the polyhydroxyalkanoate to the organic sunscreen is at least 1:2. It was surprisingly found that the composition is capable of providing improved sun protection factor.

In a second aspect, the present invention is directed to a method of providing benefit selected from the group consisting of sun protection; preventing UV damage; anti-aging; anti-blue light; blue light protection, or a combination thereof comprising the steps of applying a composition of the present invention on the desired surface.

In a third aspect, the present invention is directed to use of polyhydroxyalkanoate or a cosmetic composition comprising polyhydroxyalkanoate for providing anti-blue light and/or blue light protection benefit.

All other aspects of the present invention will more readily become apparent upon considering the detailed description and examples which follow.

### DETAILED DESCRIPTION OF THE INVENTION

Except in the examples, or where otherwise explicitly indicated, all numbers in this description indicating amounts of material or conditions of reaction, physical properties of materials and/or use may optionally be understood as modified by the word “about”.

All amounts are by weight of the composition, unless otherwise specified.

It should be noted that in specifying any range of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

Viscosities of the present invention were measured by a rheometer (Anton Paar Rheometer MCR501, Austria) system with sandblasted parallel plate (PP50), with a plate-plate gap of 0.5 mm, at a shear rate of 10 s⁻¹ and at 25° C.

“Diameter” as used herein refers to particle diameter in non-aggregated state unless otherwise stated. For polydisperse samples having particulate with diameter no greater than 1 μm, diameter means the z-average diameter measured, for example, using dynamic light scattering (see international standard ISO 13321) with an instrument such as a Zetasizer Nano™ (Malvern Instruments Ltd, UK) unless otherwise stated. For polydisperse samples having particulate with diameter no less than 1 μm, diameter means the apparent volume median diameter (D50, also known as x50 or sometimes d(0.5)) of the particles measurable for example, by laser diffraction using a system (such as a Mastersizer™ 2000 available from Malvern Instruments Ltd) meeting the requirements set out in ISO 13320 unless otherwise stated.

Preferably the polyhydroxyalkanoate is in the form of particles, more preferably the polyhydroxyalkanoate has a substantially spherical shape. Preferably the polyhydroxyalkanoate have a structure in the form of hollow sphere or porous sphere but more preferably a hollow sphere. Hollow sphere as used herein means the particle has an outer shell and a cavity inside of the outer shell to form a hollow structure. The polyhydroxyalkanoate has an average diameter of 0.1 to 60 microns, more preferably 0.2 to 30 microns, even more preferably 0.4 to 15 microns, and most preferably 1 to 10 microns.

Preferably, the polyhydroxyalkanoate has a specific surface area (BET) preferably of from 0.1 to 100 m²/g, preferably 0.5 to 30 m²/g, more preferably from 1 to 10 m²/g. “Specific surface area” as used herein refers to specific surface area determined according to Brunauer-Emmett-Teller method. The value of the specific surface area may be measured by following ASTM standard D 3663-78. Preferably, the polyhydroxyalkanoate has an oil absorption value (linseed oil) of 30 g/100 g (oil to particle) to 300 g/100 g, more preferably 50 g/100 g to 200 g/100 g and even more preferably 70 g/100 g to 130 g/100 g. The oil absorption value refers to the values measured in conformity with ASTM Method D281-84.

Preferably, the polyhydroxyalkanoate has a weight average molecular weight (Mw) of from 10,000 to 10,000,000 Da, more preferably 40,000 to 3,000,000 Da, even more preferably 150,000 to 700,000 Da.

Preferably, the polyhydroxyalkanoate comprises repeating units having the formula of:

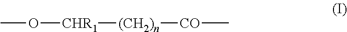

Where R₁ is selected from —H, C₁-C₁₂ alkyls, C₁-C₁₆ cycloalkyls, and C₂-C₁₂ alkenyls; n is zero or an integer of from 1 to 6. Preferably, R₁ is selected from —H, and C₁-C₆ alkyls, more preferably R₁ is methyl or ethyl. Preferably n is 1 or 2.

The polyhydroxyalkanoate may be homopolymers, or copolymers but preferred copolymer. Preferably, the polyhydroxyalkanoate comprises repeating units selected from hydroxybutyrate, hydroxyvalerate, hydroxyhexanoate, hydroxyoctanoate and hydroxyundecenenoate.

Particularly preferred polyhydroxyalkanoate comprises polyhydroxybutyrate (PHB), polyhydroxyvalerate (PHV), polyhydroxyhexanoate (PHH), polyhydroxyoctanoate (PHO), poly (hydroxybutyrate-co-hydroxyvalerate) (PHBV), poly (hydroxybutyrate-co-hydroxyexanoate) (PHBH), poly(3-hydroxybutyrate-co-4-hydroxybutyrate), poly(3-hydroxyoctanoate-co-3-hydroxyundecen-10-enoate) (PHOU), poly(3-hydroxybutyrate-co-3-hydroxyvalerate-co-4-hydroxyvalerate) (PHBW), hydroxybutyric acid/hydroxypentanoic acid copolymer, or mixtures thereof. More preferably, the polyhydroxyalkanoate comprises hydroxybutyric acid/hydroxypentanoic acid copolymer and even more preferably the polyhydroxyalkanoate is hydroxybutyric acid/hydroxypentanoic acid copolymer.

Preferably, the amount of polyhydroxyalkanoate is 0.1 to 25%, more preferably 0.5 to 22%, even more preferably 0.8 to 20%, still even more preferably 3 to 15% and most preferably 6 to 12% by weight of the composition. Preferably, the amount of hydroxybutyric acid/hydroxypentanoic acid copolymer r is 0.1 to 25%, more preferably 0.5 to 22%, even more preferably 0.8 to 20%, still even more preferably 3 to 15% and most preferably 6 to 12% by weight of the composition.

The composition comprises organic sunscreens. Sunscreens are known to absorb UV radiations and protect a surface on to which they are applied. Organic sunscreens that may be used in the present invention may be selected from UV-A, UV-B organic sunscreens and mixtures thereof. Preferably, the organic sunscreen comprises UV-B organic sunscreens.

Suitable organic sunscreens comprise 2-hydroxy-4-methoxybenzophenone, octyldimethyl p-aminobenzoic acid, digalloyltrioleate, 2,2-dihydroxy-4-methoxybenzophenone, ethyl-4-(bis(hydroxypropyl)) aminobenzoate, 2-ethylhexyl-2-cyano-3,3-diphenylacrylate, 2-ethylhexylsalicylate, glyceryl p-aminobenzoate, 3,3,5-trimethylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, 2-ethylhexyl-p-methoxycinnamate, butylmethoxydibenzoylmethane, 2-hydroxy-4-methoxybenzophenone, octyldimethyl-p-aminobenzoic acid, or mixtures thereof.

Preferably, the organic sunscreen comprises a dibenzoylmethane compound. Dibenzoylmethane compound as used herein typically refers one or more hydrogen in the benzene ring of dibenzoylmethane is replaced by C₁ to C₆ alkyl group and/or C₁ to C₆ alkoxy group. Examples of a dibenzoylmethane compound include 2-methyldibenzoylmethane, 4-isopropyldibenzoyl-methane, butyl methoxydibenzoylmethane (commercially available as Parsol® 1789 or

Avobenzone), 2,4-dimethyldibenzoylmethane, 2,5-dimethyldibenzoylmethane, 4,4′-diisopropyl-dibenzoylmethane, 2-methyl-5-isopropyl-4′-methoxydibenzoylmethane, 2-methyl-5-tert-butyl-4′-methoxy-5 dibenzoyl methane, 2,4-dimethyl-4′-methoxy dibenzoylmethane, 2,6-dimethyl-4-tert-butyl-4′-methoxy-dibenzoylmethane and mixtures thereof.

Preferably, the organic sunscreen comprises cinnamate compound. Cinnamate compound as used herein typically refers to salt, ester of cinnamic acid, one or more hydrogen in the benzene ring of cinnamic acid is replaced by C₁ to C₁₀ alkyl group and/or C₁ to C₁₀ alkoxy group. Particularly preferred cinnamate compound comprises octyl methoxycinnamate, ethoxyethyl methoxycinnamate, isoamyl methoxycinnamate, or a mixture thereof.

More preferably, the organic sunscreen comprises butyl methoxydibenzoylmethane, ethylhexyl, salicylate, octocrylene, phenylbenzimidazole sulfonic acid, octyl methoxycinnamate, or a mixture thereof. Even more preferably, the organic sunscreen comprises octyl methoxycinnamate, butyl methoxydibenzoylmethane or a mixture thereof. The most suitable organic sunscreens are 2-ethylhexyl-p-methoxycinnamate, butylmethoxydibenzoylmethane or a mixture thereof.

A safe and effective amount of organic sunscreen may be used in the compositions useful in the subject invention. The amount of the organic sunscreen is preferably 0.1% to 20%, more preferably 0.5 to 15%, even more preferably from 1% to 12%, and most preferably 1.5 to 10% of by weight of the composition.

Preferably, the weight ratio of the polyhydroxyalkanoate to the organic sunscreen is 1:2 to 15:1, more preferably 1:1.5 to 12:1, even more preferably 1:1 to 10:1, still even more preferably 1.5:1 to 6:1, and particularly preferably 1.5:1 to 3:1. Alternatively, the weight ratio of the polyhydroxyalkanoate to the organic sunscreen is preferably 3:1 to 6:1.

Preferably, the organic sunscreen comprises dibenzoylmethane compound, cinnamate compound, or a mixture thereof. Preferably, the organic sunscreen comprises dibenzoylmethane compound and cinnamate compound. Preferably, total amount of the dibenzoylmethane compound and cinnamate compound is preferably 0.1% to 10%, more preferably 0.5 to 7.5%, even more preferably from 1% to 5% by weight of the composition. Preferably, the weight ratio of the polyhydroxyalkanoate to the total amount of dibenzoylmethane compound and cinnamate compound is 1:2 to 15:1, more preferably 1:1.5 to 12:1, even more preferably 1:1 to 10:1, still even more preferably 1.5:1 to 6:1, and particularly preferably 1.5:1 to 3:1. Alternatively, the weight ratio of the polyhydroxyalkanoate to the total amount of the dibenzoylmethane compound and cinnamate compound is preferably 3:1 to 6:1.

Preferably, the organic sunscreen comprises octyl methoxycinnamate, butyl methoxydibenzoylmethane or a mixture thereof. Preferably, the organic sunscreen comprises octyl methoxycinnamate and butyl methoxydibenzoylmethane. Preferably, total amount of octyl methoxycinnamate and butyl methoxydibenzoylmethane is preferably 0.1% to 10%, more preferably 0.5 to 7.5%, even more preferably from 1% to 5% by weight of the composition. Preferably, the weight ratio of the polyhydroxyalkanoate to the total amount of octyl methoxycinnamate and butyl methoxydibenzoylmethane is 1:2 to 15:1, more preferably 1:1.5 to 12:1, even more preferably 1:1 to 10:1, still even more preferably 1.5:1 to 6:1, and particularly preferably 1.5:1 to 3:1. Alternatively, the weight ratio of the polyhydroxyalkanoate to the total amount of octyl methoxycinnamate and butyl methoxydibenzoylmethane is preferably 3:1 to 6:1.

Preferably, the present invention also provides a cosmetic composition comprising polyhydroxyalkanoate, and 0.1 to 25% of octyl methoxycinnamate by weight of the composition, wherein the weight ratio of the polyhydroxyalkanoate to octyl methoxycinnamate is at least 1:2. Preferably, the amount of octyl methoxycinnamate is preferably 0.1% to 10%, more preferably 0.5 to 7.5%, even more preferably from 1% to 5% by weight of the composition. Preferably, the weight ratio of the polyhydroxyalkanoate to octyl methoxycinnamate is 1:1.5 to 10:1, more preferably 1:1 to 8:1, even more preferably 3:1 to 6:1. Preferably, the weight ratio of the hydroxybutyric acid/hydroxypentanoic acid copolymer to octyl methoxycinnamate is 1:1.5 to 10:1, more preferably 1:1 to 8:1, even more preferably 3:1 to 6:1.

Preferably, the total amount of fatty acid and salt of fatty acid is less than 4%, more preferably less than 3% by weight of the composition. “Fatty acid” as used herein refers those carboxylic acid having a straight hydrocarbon chain of at least 5 carbon atoms. Preferably, the total amount of stearic acid and potassium stearate is less than 3%, even more preferably less than 2.5% and still even more preferably less than 2% by weight of the composition. Preferably, the composition comprises less than 2.5%, more preferably less than 2%, even more preferably 0 to 1%, still even more preferably 0 to 0.1%, 0 to 0.01% of salt of fatty acid by weight of the composition.

The composition preferably comprises a non-soap cleansing surfactant. The non-soap cleaning surfactant may be chosen from anionic, cationic, non-ionic, amphoteric surfactant and mixtures thereof. The preferred surface-active compounds that can be used are non-soap anionic, non-ionic surfactant, amphoteric surfactant or a mixture thereof.

Suitable non-soap anionic surfactants include linear alkylbenzene sulphonate, primary and secondary alkyl sulphates, particularly C₈ to C₁₅ primary alkyl sulphates; alkyl ether sulphates; olefin sulphonates; alkyl xylene sulphonates; dialkyl sulphosuccinates; fatty acid ester sulphonates; or a mixture thereof. Sodium salts are generally preferred. Most preferred non-soap anionic surfactant are linear alkylbenzene sulphonate, particularly linear alkylbenzene sulphonates having an alkyl chain length of from C₈ to C₁₅. It is preferred if the level of linear alkylbenzene sulphonate is from 0 wt % to 30 wt %, more preferably from 1 wt % to 25 wt %, most preferably from 2 wt % to 15 wt %, by weight of the total composition.

Nonionic surfactants that may be used include the primary and secondary alcohol ethoxylates, especially the C₈ to C₂₀ aliphatic alcohols ethoxylated with an average of from 1 to 20 moles of ethylene oxide per mole of alcohol, and more especially the C₁₀ to C₁₅ primary and secondary aliphatic alcohols ethoxylated with an average of from 1 to 10 moles of ethylene oxide per mole of alcohol. Non ethoxylated nonionic surfactants include alkylpolyglycosides, glycerol monoethers, and polyhydroxyamides (glucamide). It is preferred if the level of non-ionic surfactant is from 0 wt % to 30 wt %, preferably from 1 wt % to 25 wt %, most preferably from 2 wt % to 15 wt %, by weight of a fully formulated composition comprising the microcapsules of the invention.

Suitable amphoteric surfactants preferably are betaine surfactants. Examples of suitable amphoteric surfactants include, but are not limited to, alkyl betaines, alkylamido betaines, alkyl sulfobetaines, alkyl sultaines and alkylamido sultaines; preferably, those having 8 to about 18 carbons in the alkyl and acyl group. It is preferred that the amount of the amphoteric surfactant is 0 to 20 wt %, more preferably from 1 to 10 wt %, by weight of the composition.

It is also possible to include certain mono-alkyl cationic surfactants. Cationic surfactants that may be used include quaternary ammonium salts of the general formula R¹R²R³R⁴N⁺ X⁻ wherein the R groups are long or short hydrocarbon chains, typically alkyl, hydroxyalkyl or ethoxylated alkyl groups, and X is a counter-ion (for example, compounds in which R¹ is a C₈-C₂₂ alkyl group, preferably a C₈-C₁₀ or C₁₂-C₁₄ alkyl group, R² is a methyl group, and R³ and R⁴, which may be the same or different, are methyl or hydroxyethyl groups); and cationic esters (for example, choline esters).

The composition may comprise water in amount of 10 to 95% by weight of the composition, more preferably from 45 to 92%, even more preferably from 52 to 90%, most preferably from 60% to 84% by weight of the composition.

The composition may comprise resorcinol derivative. Resorcinol derivative preferably refers to that at least one hydrogen on the ring structure and/or on a hydroxy group of the resorcinol replaced with an alkyl group or phenyl alkyl group. Preferably, the resorcinol derivative is 4-substituted resorcinol. Preferably, the resorcinol derivative is selected from 4-ethyl resorcinol, 4-butyl resorcinol, 4-hexyl resorcinol, phenylethyl resorcinol, or a mixture thereof, and more preferably, the resorcinol derivative comprises 4-hexyl resorcinol. The amount of the resorcinol derivative is preferably in the range of 0.00001 to 10%, more preferably from 0.001 to 5% and most preferably from 0.1 to 0.6% by weight of the total amount of the composition.

Preferably, the composition comprises Vitamin B3 compounds (including derivatives of vitamin B3). The vitamin B3 compounds comprises niacin, nicotinic acid, niacinamide or a mixture thereof. The most preferred vitamin B3 compound is niacinamide. Amount of Vitamin B3 compounds may be 0.1 to 10%, preferably 0.5 to 5% by weight of the composition.

Preferably, the composition comprises polyhydric alcohol. Polyhydric alcohols may be selected from group of glycerin, propylyene glycol, dipropylene glycol, polypropylene glycol, polyethylene glycol, sorbitol, hydroxypropyl sorbitol, hexylene glycol, 1,3-butylene glycol, isoprene glycol, ethoxylated glycerol, propoxylated glycerol or a mixture thereof. Most preferred polyhydric alcohol is glycerol known also as glycerin. The amount of polyhydric alcohol may range anywhere from 0.1 to 20%, preferably 0.5 to 15% and more preferably 2 and 10% by weight of the composition.

Water-insoluble skin benefit agents may also be formulated into the compositions as conditioners and moisturizers. Examples include silicone oils; and vegetable triglycerides such as sunflower seed and cottonseed oils.

Some compositions may include thickeners. These may be selected from cellulosics, natural gums and acrylic polymers but not limited by this thickening agent types. Among the cellulosics are sodium carboxymethyl cellulose, hydroxyethyl cellulose, hydroxypropyl methylcellulose and combinations thereof. Suitable gums include xanthan, pectin, karaya, agar, alginate gums and combinations thereof. Among the acrylic thickeners are homopolymers and copolymers of acrylic and methacrylic acids including carbomers such as Carbopol 1382, Carbopol 982, Ultrez, Aqua SF-1 and Aqua SF-2 available from the Lubrizol Corporation. Amounts of thickener may range from 0.01 to 3% by weight of the active polymer (outside of solvent or water) in the compositions.

Preservatives can desirably be incorporated into the compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Particularly preferred preservatives are phenoxyethanol, methyl paraben, propyl paraben, imidazolidinyl urea, sodium dehydroacetate and benzyl alcohol. The preservatives should be selected having regard for the use of the composition and possible incompatibilities between the preservatives and other ingredients. Preservatives are preferably employed in amounts ranging from 0.01% to 2% by weight of the composition.

A variety of other optional materials may be formulated into the compositions. These may include: antimicrobials such as 2-hydroxy-4,2′,4′-trichlorodiphenylether (triclosan), 2,6-dimethyl-4-hydroxychlorobenzene, and 3,4,4′-trichlorocarbanilide; scrub and exfoliating particles such as polyethylene and silica or alumina; cooling agents such as menthol; skin calming agents such as aloe vera; and colorants.

In addition, the compositions of the invention may further include 0.5 to 10% by weight of sequestering agents, such as tetra sodium ethylenediaminetetraacetate (EDTA), EHDP or mixtures; opacifiers and pearlizers such as ethylene glycol distearate, titanium dioxide or Lytron 621 (Styrene/Acrylate copolymer); all of which are useful in enhancing the appearance or properties of the product.

The viscosity of the composition is preferably from 1 to 500 Pa·s, more preferably from 5 to 150 Pas, even more preferably from 10 to 60 Pas, and most preferably from 20 to 40 Pa·s. Viscosities as used herein were measured by a rheometer (Anton Paar Rheometer MCR501, Austria) system with sandblasted parallel plate (PP50), with a plate-plate gap of 0.5 mm, at a shear rate of 10 s⁻¹ and at 25° C. Preferably, the composition is an emulsion, more preferably an oil-in-water emulsion. Preferably the composition is a fluid liquid at 25° C. and atmospheric pressure.

Preferably, the cosmetic composition is a skin care composition. Skin care composition refers to a composition suitable for topical application to human skin, including leave-on and wash-off products but preferably leave-on compositions. The term “leave-on” as used with reference to compositions herein means a composition that is applied to or rubbed on the skin, and left thereon. The term “wash-off” as used with reference to compositions herein means a skin cleanser that is applied to or rubbed on the skin and rinsed off substantially immediately subsequent to application. The term “skin” as used herein includes the skin on the face, neck, chest, abdomen, back, arms, under arms, hands, and legs. Preferably “skin” means includes the skin on the face and under arms, more preferably skin means skin on the face other than lips and eyelids. The composition is particularly preferably a moisturizer rather than a make-up product.

Preferably, the composition is a topical composition. Preferably, the composition may be in the form of cream, lotion, ointment, solution, suspension, emulsion, paste, gel, powder, powder foundation, emulsion foundation, wax foundation, or spray. More preferably, the composition may be formulated in the form of cream, lotion, ointment, emulsion, gel, or a spray.

The present invention also provided a method of providing benefit of anti-blue light and/or blue light protection comprising the steps of applying polyhydroxyalkanoate or a composition comprising polyhydroxyalkanoate on the desired surface. The present invention also provided use of a composition of the present invention for providing benefit selected from the group consisting of sun protection; preventing UV damage; anti-aging, or a combination thereof.

For sake of clarity, the composition comprises polyhydroxyalkanoate typically provides a greater benefit of anti-blue light and/or blue light protection in comparison to same composition comprises no polyhydroxyalkanoate.

The present invention also provides a cosmetic composition comprises polyhydroxyalkanoate for use in providing benefit selecting from anti-blue light and blue light protection. The present invention also provided use of polyhydroxyalkanoate in a cosmetic composition for providing benefit of anti-blue light and/or blue light protection.

Preferably the use is non-therapeutic. Preferably the method is non-therapeutic. The term non-therapeutic typically means for cosmetic purposes and not curative or therapeutic purposes.

Preferably, the composition is capable of providing at least 1.2 times, more preferably 1.3 to 4 times, even more preferably 1.6 to 3 times of SPF increase, compared with same composition but does not contain polyhydroxyalkanoate.

The individual Sun Protection Factor (SPF) for each subject is defined as the ratio of the amount of energy (dose, in units of J/m² or in seconds of exposure time) required to produce minimal erythema on protected skin to the amount of energy needed to produce minimal erythema on untreated skin calculated as follows:

SPF=(Minimal Erythema Dose (MED) of Protected Skin)/(MED Unprotected of Control Site)

The SPF for the composition may be calculated by taking the arithmetic mean of the individual SPF values for all the subjects.

Preferably, the composition is capable of providing at least 10%, more preferably 15 to 75%, even more preferably 20 to 60% of decrease in average transmittance value at wavelength of 400 to 450 nm, compared with same composition but does not contain polyhydroxyalkanoate. The average transmittance value for each sample at wavelength of 400 to 450 nm may be obtained by number averaging the tested transmittance values among 400 to 450 nm at an interval of 1 nm.

The following examples are provided to facilitate an understanding of the invention. The examples are not intended to limit the scope of the claims.

### EXAMPLES

### Example 1

This example demonstrates that sun protection factor was significantly increased by including polyhydroxyalkanoate and organic sunscreen with specific weight ratios and amounts according to the present invention and that polyhydroxyalkanoate provided blue light protection benefit.

TABLE 1

| | Sample (wt %)* | | | | | | | |
|---|---|---|---|---|---|---|---|---|
| Ingredient | A | B | C | D | 1 | E | 2 | F |
| Ecofil B110 ^{a} | 8.0 | — | 8.0 | — | 8.00 | — | 8.0 | 0.6 |
| Ethylhexyl | — | — | — | 2.25 | 2.25 | 2.25 | 2.25 | 2.25 |
| methoxycinnamate ^{b} | | | | | | | | |
| Butyl | — | — | — | — | — | 1.2 | 1.2 | — |
| methoxydibenzoylmethane ^{c} | | | | | | | | |
| Titanium dioxide ^{d} | — | 3.0 | 3.0 | — | — | — | — | — |
| Polyoxyethylene (20) | 2.0 | 2.0 | 2.0 | 2.0 | 2.0 | 2.0 | 2.0 | 2.0 |
| Sorbitan monolaurate | | | | | | | | |
| Glycerin | 2.0 | 2.0 | 2.0 | 2.0 | 2.0 | 2.0 | 2.0 | 2.0 |
| Simulgel EG ^{e} | 1.5 | 1.5 | 1.5 | 1.5 | 1.5 | 1.5 | 1.5 | 1.5 |
| Dimethicone | 6.0 | 6.0 | 6.0 | 6.0 | 6.0 | 6.0 | 6.0 | 6.0 |
| Caprylic/capric triglyceride | 2.0 | 2.0 | 2.0 | 2.0 | 2.0 | 2.0 | 2.0 | 2.0 |
| Phenoxyethanol | 0.5 | 0.5 | 0.5 | 0.5 | 0.5 | 0.5 | 0.5 | 0.5 |
| Water | to 100 | to 100 | to 100 | to 100 | to 100 | to 100 | to 100 | to 100 |

* The level in this table refers the level of left-listed ingredients, unless otherwise specified.
^{a} Ecofil B110, supplied by Eckart, containing 100% of Hydroxybutyric acid/Hydroxypentanoic acid copolymer.
^{b} PASCOL ® MCX, supplied by DSM, containing 100% of Ethylhexyl Methoxycinnamate.
^{c} PARSOL ® 1789, supplied by DSM, containing 100% of Butyl methoxydibenzoylmethane.
^{d} MT-100Z, supplied by Tayca, containing 100% of titanium dioxide.
^{e} Simulgel EG, supplied by Seppic, having INCI Name of Sodium Acrylate/Sodium Acryloyldimethyl Taurate Copolymer (and) Isohexadecane (and) Polysorbate 80.

The samples in Table 1 were formulated by following standard procedure for skin care composition.

Thin film transmittance measurements were done using Labsphere's Ultraviolet transmittance analyzer. PMMA plate with ~6 μm roughness from Schonberg GmbH & Co were used. Then, 4 mg/cm² of sample was applied on PMMA plate, distributed and spread uniformly. The drying time for the PMMA plates was 30 minutes. After the drying time, sample plates were exposed to UV light and transmittance scan was recorded. This scan gives the transmittance as a function of wavelength (290-450 nm) for a given sample. For a single plate nine different spots were scanned. The reference transmittance scan was obtained using a blank plate, with no sample on the PMMA plates with glycerin spread on it. The transmittance values were used to arrive at the SPF values using the UV-2000s application provided with the instrument. The average transmittance value for each sample at wavelength of 400 to 450 nm (blue light) was also obtained by number averaging the tested transmittance values among 400 to 450 nm.

Table 2 shows the results of the SPF and average transmittance value at wavelength of 400 to 450 nm for samples of Table 1.

TABLE 2

| | Ecofil | PASCOL ® | PARSOL ® | | Weight ratio of | | Average |
|---|---|---|---|---|---|---|---|
| | B110 | MCX | 1789 | MT-100Z | Ecofil B110 | | Transmittance |
| Sample | (wt %) | (wt %) | (wt %) | (wt %) | to sunscreen | SPF | (%) |
| A | 8.0 | — | — | — | — | 1.31 | 78.0 |
| B | — | — | — | 3.0 | — | 9.60 | 66.6 |
| C | 8.0 | — | — | 3.0 | — | 7.63 | 47.0 |
| D | — | 2.25 | — | — | — | 4.62 | 108.6 |
| 1 | 8.0 | 2.25 | — | — | 3.56:1 | 10.09 * | 58.6 |
| E | — | 2.25 | 1.2 | — | — | 5.3 | 107.8 |
| 2 | 8.0 | 2.25 | 1.2 | — | 2.32:1 | 13.7 * | 57.6 |
| F | 0.6 | 2.25 | — | — | 1:3.75 | 4.69 | — |

* Significantly higher than that sum of SPF of sample A and D.
** Significantly higher than that sum of SPF of sample A and E.

It is evident from Table 2 that the samples of the present invention provided significant higher SPF (1 vs. A and D; 2 vs. A and E). In contrast, when the weight ratio of polyhydroxyalkanoate to organic sunscreen was decreased to the scope outside the range of the present invention, or inorganic sunscreen was used, the SPF was not able to be elevated. In addition, the times of SPF increase for sample 2 (SPF (sample 2)/SPF (Sample E)) is higher than sample 1 (SPF (sample 1)/SPF (Sample D)). In addition, the average transmittance value at wavelength of 400 to 450 nm showing that the compositions A, B, C, 1 and 2 is capable of providing the benefit of anti-blue light and blue light protection.

### Example 2

This example demonstrates that sun protection factor was significantly increased by combining polyhydroxyalkanoate with organic sunscreens.

TABLE 3

Sample (wt %)*

| | G | 3 |
|---|---|---|
| Ecofil B110 ^{a} | — | 8.00 |
| Butyl Methoxydibenzoylmethane ^{b} | 1.00 | 1.00 |
| Ethylhexyl Salicylate | 4.00 | 4.00 |
| Octocrylene | 1.00 | 1.00 |
| Phenylbenzimidazole Sulfonic Acid | 1.50 | 1.50 |
| Disodium EDTA | 0.10 | 0.10 |
| Carbopol Ultrez 10 | 0.40 | 0.40 |
| Glycerin | 2.50 | 2.50 |
| Xanthan Gum | 0.30 | 0.30 |
| Potassium Hydroxide | 0.54 | 0.54 |
| Sodium Hydroxide | 0.22 | 0.22 |
| Dimethicone | 1.00 | 1.00 |
| Glycol Stearate & Stearamide AMP | 1.38 | 1.38 |
| Peg-100 Stearate | 1.18 | 1.18 |
| Cetyl Alcohol | 0.37 | 0.37 |
| Glyceryl Stearate | 0.64 | 0.64 |
| Stearic Acid | 2.35 | 2.35 |
| Caprylyl Glycol | 0.50 | 0.50 |
| Phenoxyethanol | 0.40 | 0.40 |
| Water | to 100 | to 100 |

*The level in this table refers the level of left-listed ingredients, unless otherwise specified.
^{a} Ecofil B110, supplied by Eckart, containing 100% of Hydroxybutyric acid/ Hydroxypentanoic acid copolymer.
^{b} PARSOL ® 1789, supplied by DSM, containing 100% of Butyl methoxydibenzoylmethane.

The samples in Table 3 were formulated by following standard procedure for skin care composition.

Similar tests were conducted to obtain the SPF except that 2 mg/cm² of samples of Table 3 was applied on PMMA plate, and the transmittance scan was repeated for 3 plates and the data reported is thus an average of 27 readings.

TABLE 4

| | Weight ratio of Ecofil | |
|---|---|---|
| Sample | B110 to sunscreen | SPF |
| G | — | 23.5 |
| 3 | 1.07:1 | 32.2 |

*Significantly higher than that sum of SPF of sample A and G.

Table 4 has demonstrated that the samples according to the present invention provided 5 significant higher SPF. Moreover, the times of SPF increase for sample 3 (SPF (sample 3)/SPF (sample G)) is lower than either that for sample 1 or that for sample 2.

1. A cosmetic composition comprising:
(a) a polyhydroxyalkanoate; and
(b) 0.1 to 25% of organic sunscreen by weight of the composition;
wherein the polyhydroxyalkanoate to the organic sunscreen is present in a weight ratio of at least 1:2.

2. The composition according to claim 1, wherein the polyhydroxyalkanoate has an average diameter of 0.2 to 30 microns.

3. The composition according to claim 1, wherein the polyhydroxyalkanoate has an oil absorption value of 30 g/100 g to 300 g/100 g.

4. The composition according to claim 1, wherein the polyhydroxyalkanoate has a weight average molecular weight of from 40,000 to 3,000,000 Da.

5. The composition according to claim 1, wherein the polyhydroxyalkanoate comprises hydroxybutyric acid/hydroxypentanoic acid copolymer.

6. The composition according to claim 1, wherein the polyhydroxyalkanoate is present in an amount of from 0.1 to 25% by weight of the composition.

7. The composition according to claim 1, wherein the sunscreen organic comprises butyl methoxydibenzoylmethane, ethylhexyl, salicylate, octocrylene, phenylbenzimidazole sulfonic acid, octyl methoxycinnamate, or a mixture thereof.

8. The composition according to claim 1, wherein the organic sunscreen is present in an amount of from 0.5 to 15% by weight of the composition.

9. The composition according to claim 1, wherein the polyhydroxyalkanoate to the organic sunscreen is present in a weight ratio of from 1:2 to 15:1.

10. The composition according to claim 1, wherein the organic sunscreen comprises dibenzoylmethane compound, cinnamate compound, or a mixture thereof.

11. The composition according to claim 10, wherein the organic sunscreen comprises octyl methoxycinnamate, butyl methoxydibenzoylmethane, or a mixture thereof.

12. The composition according to claim 10, wherein the polyhydroxyalkanoate to a total amount of dibenzoylmethane compound and cinnamate compound is present in a weight ratio of from 1:1 to 10:1.

13. The composition according to claim 1, wherein the composition comprises less than 2.5% of salt of fatty acid by weight of the composition.

14. A method of providing benefit selected from the group consisting of sun protection; preventing UV damage; anti-aging; anti-blue light; blue light protection, or a combination thereof comprising the steps of applying a composition according to claim 1 on the desired surface.

15. (canceled)

16. The composition according to claim 1, wherein the polyhydroxyalkanoate has an average diameter of 1 to 10 microns.

17. The composition according to claim 1, wherein the polyhydroxyalkanoate is present in an amount of from 0.8 to 20% by weight of the composition.

18. The composition according to claim 1, wherein the polyhydroxyalkanoate is present in an amount of from 3 to 15% by weight of the composition.

19. The composition according to claim 1, wherein the organic sunscreen is present in an amount of from 1.5 to 10% by weight of the composition.

20. The composition according to claim 1, wherein the polyhydroxyalkanoate to the organic sunscreen is present in a weight ratio of from 1.5:1 to 3:1.

21. The composition according to claim 10, wherein the polyhydroxyalkanoate to a total amount of dibenzoylmethane compound and cinnamate compound is present in a weight ratio of from 1.5:1 to 6:1.
